(19) United States
(12) Patent Application Publication
(10) Pub. No.: US 2026/0131071 A1
(43) Pub. Date: May 14, 2026

(54) INFUSION PUMP SYSTEM WITH SIMPLIFIED FALLBACK CLOSED LOOP ALGORITHM

(71) Applicant: Tandem Diabetes Care, Inc, San Diego, CA (US)

(72) Inventor: Paul Harris, San Diego, CA (US)

(21) Appl. No.: 19/379,147

(22) Filed: Nov. 4, 2025

Related U.S. Application Data
(60) Provisional application No. 63/719,288, filed on Nov. 12, 2024.

(51) Int. Cl. A61M 5/142

(52) U.S. Cl. CPC A61M 5/14244; A61M 2005/14208; A61M 2202/0007; A61M 2202/04; A61M 2205/3303; A61M 2205/3327; A61M 2205/3576; A61M 2205/50; A61M 2230/201

(57) ABSTRACT

Methods and systems are configured for maintaining closed loop therapy in circumstances where a phone or other user device providing therapy commands to the pump according to a closed loop therapy algorithm becomes disconnected from the pump. A fallback or backup closed loop algorithm can be stored on the pump that may be a simplified version of the closed loop algorithm operated on the phone having lower memory and computational requirements such that it can be operated on the pump. The fallback algorithm may also require less battery power to be operated. If the pump detects that the connection with the phone has been lost, the pump can establish communications with the CGM to receive the glucose levels (if not already connected). The pump can then activate the fallback close loop algorithm stored on the pump and make therapy determinations based on the CGM data according to the fallback closed loop algorithm.

### PRIORITY CLAIM

The present application claims the benefit of U.S. Provisional Application No. 63/719,288 filed November 12, 2024, which is hereby incorporated herein by reference in its entirety.

### TECHNICAL FIELD

The present invention is directed to ambulatory infusion pumps and more particularly to operation of ambulatory infusion pumps in a closed-loop or semi-closed-loop manner.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with Type 1 or Type 2 diabetes. Some insulin injecting pumps are configured as portable or ambulatory infusion devices that can provide continuous subcutaneous insulin injection and/or infusion therapy as an alternative to multiple daily injections of insulin via a syringe or an insulin pen. Such pumps can be worn or carried by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Patent Nos. 8,287,495; 8,573,027; 8,986,253; and 9,381,297, each of which is incorporated herein by reference in its entirety.

Ambulatory infusion pumps have generally been controlled by a user interface provided on the pump. With the proliferation of handheld electronic devices, such as mobile phones (e.g., smartphones), there is a desire to be able to remotely utilize such devices, as well as dedicated wireless controllers designed to work with one or more infusion pumps and/or types of infusion pumps, to optimize usage of infusion pumps. These remote controllers would enable a pump to be monitored, programmed and/or operated more privately, more conveniently and more comfortably. Accordingly, one potential use of dedicated remote devices and handheld consumer electronic devices (such as smartphones, tablets and the like) is to utilize such devices as controllers for remotely programming and/or operating infusion pumps.

Use of such devices to communicate with and/or control infusion pumps has also provided an ability to utilize more complex therapy control algorithms. For example, some infusion pump systems can utilize feedback from a continuous glucose monitor (CGM) or other device providing glucose levels of the user to automatically adjust therapy based on glucose levels in a closed loop manner. Such closed loop algorithms can be complex and require a large amount of memory, computational power and battery power. As such, it can be beneficial to utilize a user’s phone or other handheld electronic device to operate the algorithm because such devices generally have greater memory, computational power and battery life than an ambulatory infusion pump. However, if the electronic device loses the connection with the infusion pump the pump can no longer receive the closed loop therapy commands from the phone. In circumstances where such a connection is lost, infusion pumps revert to open loop therapy that is not based on CGM data. Typically, upon initiating open loop therapy the pump will deliver a preprogrammed basal rate stored in memory, which may not be optimal for a user’s needs at a given time.

### SUMMARY

Disclosed herein are methods and systems for maintaining closed loop therapy in circumstances where a phone or other user device providing therapy commands to the pump according to a closed loop therapy algorithm becomes disconnected from the pump. A fallback or backup closed loop algorithm can be stored on the pump that may be a simplified version of the closed loop algorithm operated on the phone having lower memory and computational requirements such that it can be operated on the pump. The fallback algorithm may also require less battery power to be operated. If the pump detects that the connection with the phone has been lost, the pump can establish communications with the CGM to receive the glucose levels (if not already connected). The pump can then activate the fallback closed loop algorithm stored on the pump and make therapy determinations based on the CGM data according to the fallback closed loop algorithm.

In an embodiment, a method of providing diabetes therapy can include delivering medicament to a user with an ambulatory infusion pump according to therapy commands received from a remote electronic device of a user calculated with a primary closed loop therapy algorithm based on glucose levels of a user from a continuous glucose monitoring device. It may be detected that communications between the ambulatory infusion pump and the remote electronic device have been interrupted. Glucose levels of the user can then be received from the continuous glucose monitoring device at the ambulatory infusion pump. A fallback closed loop therapy algorithm operating on the ambulatory infusion pump can be activated in response to communications between the ambulatory infusion pump and the remote electronic device being interrupted. The fallback closed loop therapy algorithm can calculate therapy commands for delivery of medicament to the user with the ambulatory infusion pump based on the glucose levels received from the continuous glucose monitoring device at the ambulatory infusion pump.

In an embodiment, an ambulatory infusion pump can include a pump mechanism configured to facilitate delivery of insulin to a user and a communications interface configured to receive communications from a remote electronic device. At least one processor can be configured to receive therapy commands from the remote electronic device calculated with a primary closed loop algorithm based on glucose levels of a user from a continuous glucose monitoring device. If it is detected that communications with the remote electronic device have been interrupted the glucose levels of the user from the continuous glucose monitoring device can be received and a fallback closed loop therapy algorithm can be activated in response to communications with the remote electronic device being interrupted. The fallback closed loop therapy algorithm can calculate therapy commands for delivery of medicament to the user based on the glucose levels received from the continuous glucose monitoring device.

In embodiments, an ambulatory infusion pump system can include a remote electronic device and an ambulatory infusion pump. The remote electronic device can be configured to receive glucose levels of a user from a continuous glucose monitoring device and to calculate therapy commands with a primary closed loop algorithm based on the glucose levels. The ambulatory infusion pump can be configured to receive the therapy commands from the remote electronic device and to deliver insulin to the user according to the therapy commands. The ambulatory infusion pump can be further configured to detect that communications with the remote electronic device have been interrupted. In response, the glucose levels of the user can be received from the continuous glucose monitoring device at the ambulatory infusion pump and a fallback closed loop therapy algorithm can be activated. The fallback closed loop therapy algorithm can calculate therapy commands for delivery of medicament to the user based on the glucose levels received from the continuous glucose monitoring device.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 depicts an embodiment of an infusion pump system according to the disclosure.

FIG. 2 depicts a block diagram representing an embodiment of an infusion pump system according to the disclosure.

FIGS. 3A-3B depict an embodiment of an infusion pump system according to the disclosure.

FIG. 4 depicts an embodiment of an infusion pump system according to the disclosure.

FIGS. 5A-5B depict remote control devices for an infusion pump system according to embodiments of the disclosure.

FIG. 6 depicts a schematic representation of an infusion pump system according to an embodiment of the disclosure.

FIG. 7 depicts a flowchart of method steps for a method of providing therapy with an ambulatory infusion pump according to the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

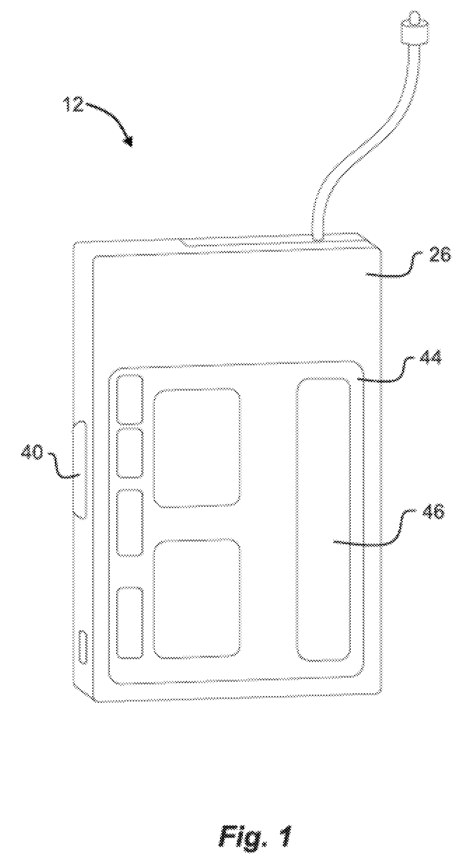

FIG. 1 depicts an exemplary medical device that can be used with embodiments of the disclosure. In this embodiment, the medical device is configured as a pump 12, such as an infusion pump, that can include a pumping or delivery mechanism and reservoir for delivering medicament to a patient and an output/display 44. The type of output/display 44 may vary as may be useful for a particular application. The output/display 44 may include an interactive and/or touch sensitive screen 46 having an input device such as, for example, a touch screen comprising a capacitive screen or a resistive screen. The pump 12 may additionally include a keyboard, microphone, or other input device known in the art for data entry, which may be separate from the display. The pump 12 may also include a capability to operatively couple to one or more blood glucose meters (BGMs) or continuous blood glucose monitors (CGMs) and/or one or more secondary devices such as a remote display, a remote control device, a laptop computer, personal computer, tablet computer, a mobile communication device such as a smartphone, a wearable electronic watch, smart ring, electronic health or fitness monitor, or personal digital assistant (PDA), a CGM display etc.

In one embodiment, the medical device can be a portable pump configured to deliver insulin to a patient. Further details regarding such pump devices can be found in U.S. Patent No. 8,287,495, which is incorporated herein by reference in its entirety. In other embodiments, the medical device can be an infusion pump configured to deliver one or more additional or other medicaments to a patient.

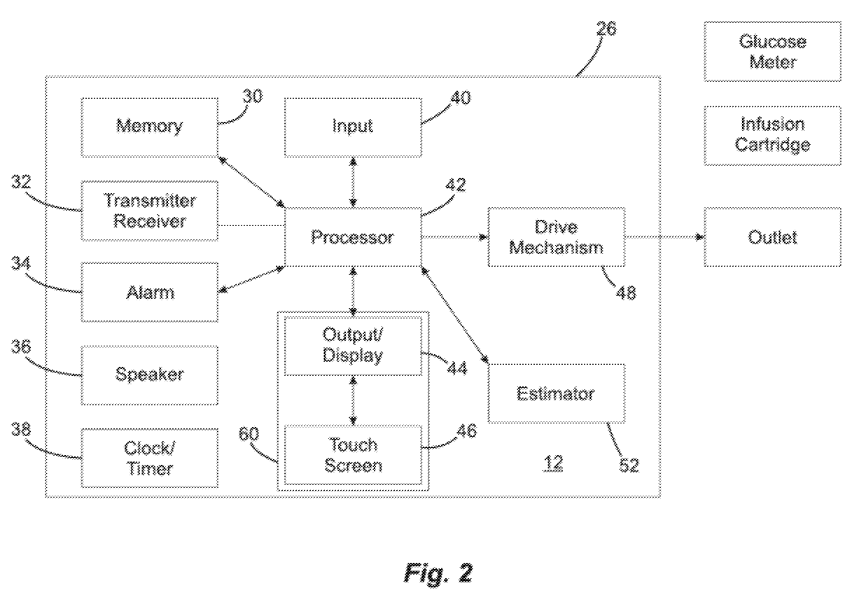

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52, and a microphone (not pictured). One embodiment of a user interface as shown in FIG. 2 is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor 42 to receive signals and/or other data from one or more input devices, such as sensors that may sense pressure, temperature and/or other parameters.

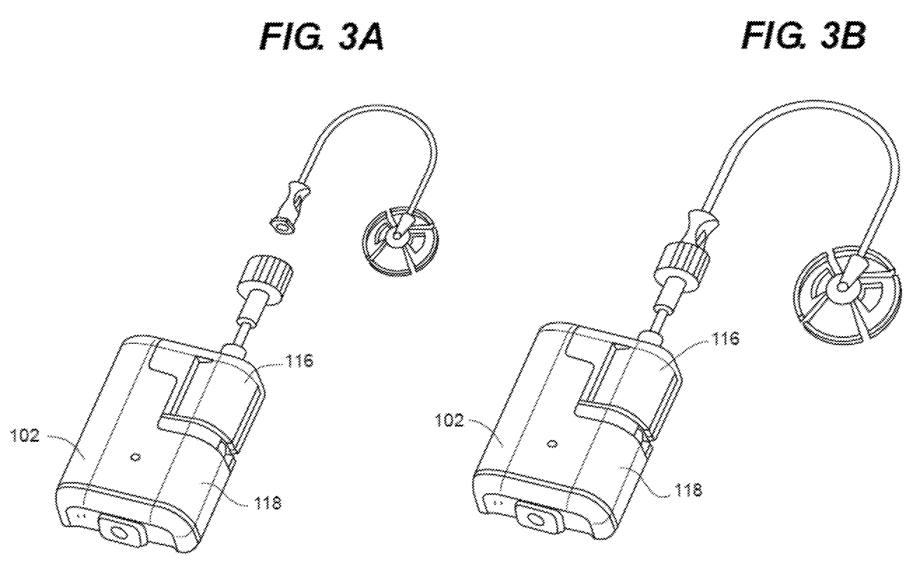

FIGS. 3A-3B depict a second infusion pump that can be used in conjunction with one or more embodiments of the ambulatory infusion pump system of the present disclosure. Pump 102 includes a pump drive unit 118 and a medicament cartridge 116. Pump 102 includes a processor 42 that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, smart ring, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receives commands from a separate device for control of some or all of the operations of the pump. Such separate device can include, for example, a dedicated remote device or a consumer electronic device such as a smartphone having a processor executing an application configured to enable the device to transmit operating commands to the processor 42 of pump 102. In some embodiments, processor 42 can also transmit information to one or more separate devices, such as information pertaining to device parameters, alarms, reminders, pump status, etc. Such separate device can include any remote display, remote device, remote control, or a consumer electronic device as described previously.

Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Patent No. 10,279,106 and U.S. Patent Publication Nos. 2016/0339172 and 2017/0049957, each of which is hereby incorporated herein by reference in its entirety.

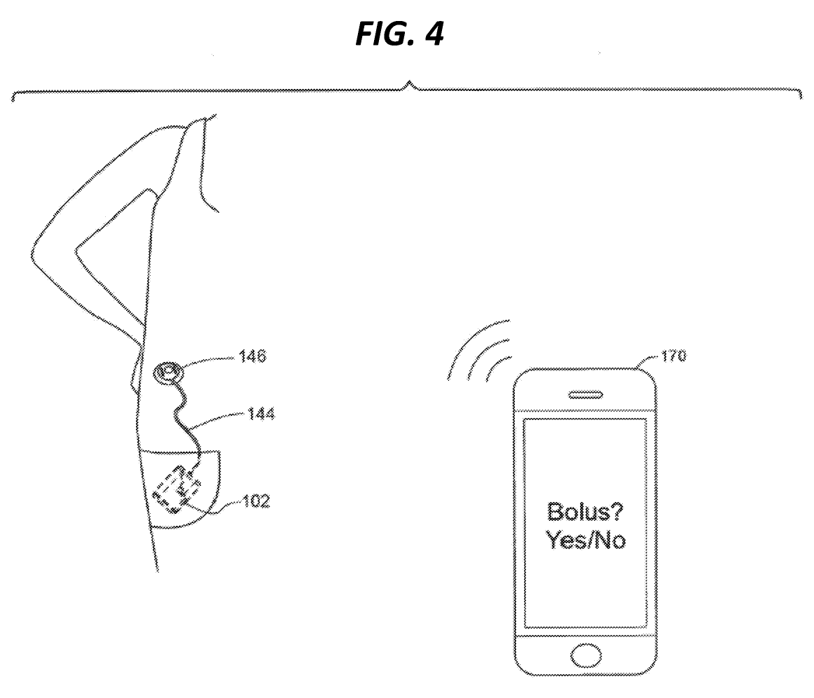

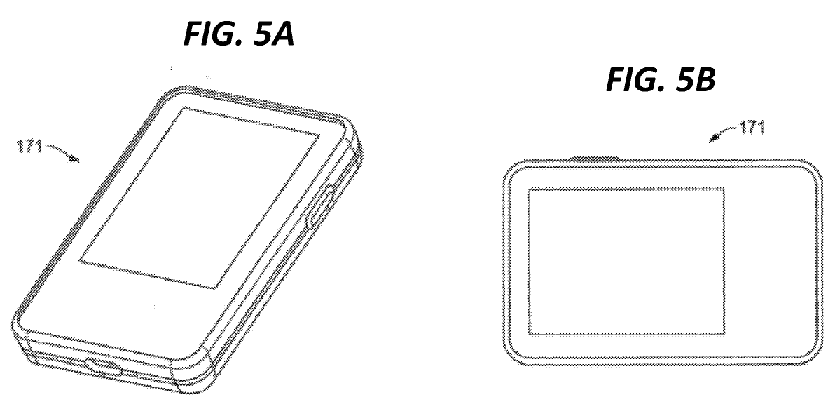

Referring to FIGS. 4-5B, one or more remote devices 170, 171 can be used to communicate with the processor 42 of pump 12 and/or pump 102 to control delivery of medicament and transfer data with pump 12/102 via a wired or a wireless electromagnetic signal, such as via, e.g., a near field communication (NFC) radio frequency (RF) modality or other RF modalities such as Bluetooth®, Bluetooth® low energy, mobile or Wi-Fi communication protocols, for example, according to embodiments of the present disclosure. Such a remote device can include, for example, a mobile communication device 170, such as a smart phone (as depicted in FIG. 4) executing a software application for control of the pump 12/102, a dedicated remote controller 171 (as depicted in FIGS. 5A-5B), a wearable electronic watch, smart ring, electronic health or fitness monitor or personal digital assistant (PDA), etc., or a tablet, laptop or personal computer. Such communications between (and among) the one or more remote devices 170, 171 and pump 12/102 may be one-way or two-way for, e.g., effective transfer of data among the devices and the pump, control of pump operations, updating software on the devices and/or pump, and allowing pump-related data to be viewed on the devices and/or pump.

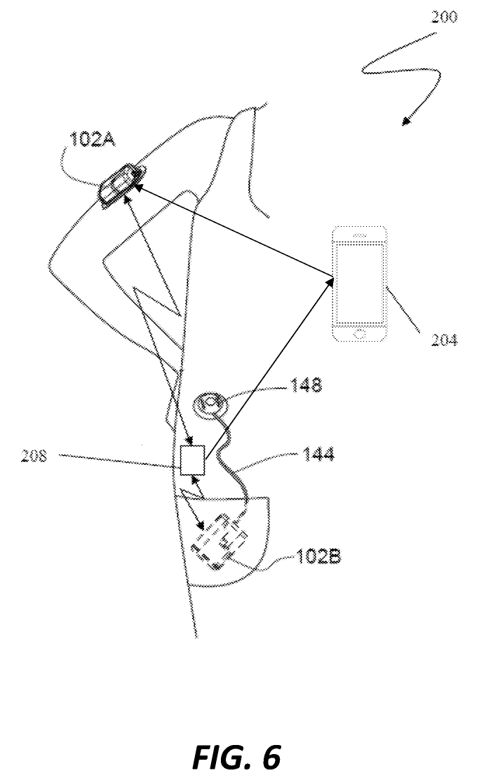

FIG. 6 depicts a schematic representation of a pump system 200 according to an embodiment of the disclosure. System 200 includes a user-wearable infusion pump such as pump 12 or pump 102 described above. In embodiments, a user can alternatively wear the pump 102A directly on the body or place the pump 102B in the user's pocket or other location near the body with infusion tubing 144 extending to an infusion set 148 on the user's body. The system 200 also includes a continuous glucose monitoring (CGM) sensor with a corresponding transmitter 208. The CGM sensor obtains measurements relating to glucose levels in the body and the transmitter can communicate that information to pump 102A/B. Pump 208 can then use the glucose data in making therapy determinations.

The system can also include one or more devices such as a smartphone 204 or other multi-purpose consumer electronic device capable of operating a software application to communicate with and/or control the pump and, alternatively or additionally, a dedicated remote device designed specifically for use with pump 102A/102B. The smartphone 204 or other remote electronic device can in some embodiments also be capable of communication with CGM sensor/transmitter 208. In some embodiments, due to either computational and/or memory constraints of the pump 102A/102B and/or to conserve battery of the pump 102A/102B, the smartphone 204 may communicate with the CGM sensor 208 to obtain the measured glucose levels, analyze glucose levels with a closed loop therapy algorithm, and transmit therapy commands to the pump 102A/102B determined by the closed loop therapy algorithm.

Although depicted with the multi-purpose consumer electronic device 204 being a smartphone, in various embodiments the consumer electronic device can alternatively or additionally include one or more of a wearable electronic watch, such as a smartwatch, a smart ring, electronic health or fitness monitor, personal digital assistant (PDA), or a tablet, laptop or personal computer, etc. A multi-purpose consumer electronic device 204 can be any device sold to consumers and used for a variety of functions and which can be configured or programmed to communicate with and/or control an infusion pump as one of said functions. In some embodiments, systems as described herein may include more than one multi-purpose consumer electronic device 204 configured for communication with the infusion pump (e.g., a smartphone and a smart watch).

As noted above, in systems such as the system 200 of FIG. 6, a closed loop algorithm for determining therapy to be delivered with pump 102A/102B may operate on the smartphone 204 or other user device. This may be due to the complexity of the algorithm requiring computational and memory capacity that is too great for the pump and/or in order to offload therapy calculations and/or CGM communications to the smartphone to preserve the pump’s battery. However, if communications between the phone 204 and the pump 102A/102B are interrupted, the pump will not receive therapy commands according to the closed loop algorithm. The standard approach in this circumstance is to revert to delivering medicament according to a stored open loop basal profile for the user. However, a user may have outdated basal rates not reflective of the user’s current medicament needs or may have current glucose levels that could become dangerously low or high if the open loop basal rates are delivered.

Embodiments disclosed herein therefore provide for a mechanism to maintain closed loop therapy based on glucose levels from the CGM when communications with a phone operating a closed loop algorithm and a pump are interrupted. In embodiments, a fallback or backup closed loop algorithm can be stored on the pump that may be a simplified version of the closed loop algorithm operated on the phone having lower memory and computational requirements such that it can be operated on the pump and/or requiring less battery power to be operated. If the pump detects that the connection with the phone has been lost, the pump can establish communications with the CGM to receive the glucose levels (if not already connected). The pump can then activate the fallback closed loop algorithm stored on the pump and make therapy determinations based on the CGM data according to the fallback closed loop algorithm.

The fallback closed loop algorithm can be simplified relative to the closed loop algorithm executed by the phone in one or more of a number of different ways. For example, the algorithm executed by the phone may use mathematical calculations or a model such as machine learning, a neural network, predictive dosing simulations, etc. that is too complex for operation on the pump that is not utilized in the algorithm on the pump. The algorithm executed by the phone may require computational resources (i.e., processing and memory resources) that are greater or otherwise distinct from computational resources available on the pump. For example, the algorithm executed by the phone may require more random access memory (RAM) and/or tera floating-point operations per second (TFLOPS) than available on the pump. The algorithm executed by the pone may require specialized processor architecture such as a certain number of cores, parallel computer architecture, and/or a tensor processing unit. The algorithm on the phone may also access and use significantly more data, e.g., from the cloud, that is not utilized in the algorithm on the pump. The algorithm on the phone may utilize additional sensors and/or input such as, for example, heart rate, that is not utilized in the algorithm on the pump. The algorithm on the phone may be continually updated to be personalized for the user, whereas the algorithm on the pump may remain the same and not continually adapt to the user.

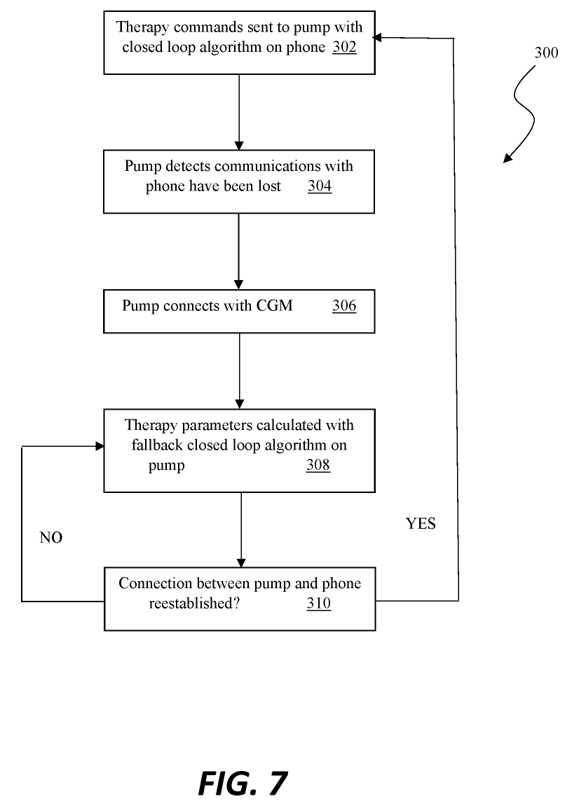

FIG. 7 depicts a flowchart of method steps for a method of 300 providing therapy with an ambulatory infusion pump according to the disclosure. At step 302, therapy commands are being sent to the pump based on calculations made according to a closed loop algorithm operating on a user’s phone or other device based on glucose levels received at the phone from a CGM. At step 304, the pump determines that communications with between the pump and the phone have been interrupted. The pump then establishes communications with the CGM at step 306, if the devices are not already connected. This can be done immediately upon determining that communications with the phone have been lost or after a predetermined amount of time of waiting for the phone to reestablish a connection with the pump. A fallback closed loop algorithm on the pump, which may be a simplified version of the algorithm operated by the phone, can then be used at step 308 to determine therapy parameters for the pump based on the glucose levels. The fallback algorithm may be simplified by requiring fewer computational resources (i.e., processing and memory resources) than the algorithm executed on the phone. For example, the fallback algorithm may require less RAM and/or TFLOPS than the algorithm operated by the phone. The fallback algorithm may be executable without specialized processor architecture such as a certain number of cores, parallel computer architecture, and/or a tensor processing unit. By being executable with fewer computational resources, the fallback algorithm may be more energy efficient and enable a smaller form factor for the pump (e.g., from a smaller battery and computational components). At step 310 it is determined if the connection between the pump and the phone has been reestablished. If not, the pump will continue to employ the fallback closed loop algorithm. If communications between the pump and the phone have been reestablished, the method reverts to step 302 with therapy commands being determined by the algorithm on the phone and being sent from the phone to the pump.

In addition to communication interruptions, the system can transition between employing the algorithm on the phone and the algorithm on the pump for various other reasons.

For example, if the battery level of the phone falls below a low power threshold, the system may automatically transition to the algorithm on the pump to ensure that there is no lapse in closed loop operation if the phone’s battery dies. In embodiments, this could occur automatically when the phone issues a low battery alert at a certain level such as at 20%, 15%, 10%, 5%, etc. The system could then revert to use of the algorithm on the phone when the phone is connected to a power source and/or reaches a threshold battery level.

A user may also be able to manually switch between algorithms for various reasons. For example, the user may find that the more complex algorithm on the phone works better for glycemic control during the user’s day to day activities, but that the user prefers the simplified algorithm on the pump when the user is exercising. In such an example, the user may be able to manually activate the algorithm on the pump when the user is going to exercise and then reactivate the algorithm on the phone following the exercise. Similarly, if the system includes a smart watch or fitness monitor the user could preprogram the system to switch algorithms automatically if it is detected that the user is exercising.

In addition, the algorithm on the pump can in some embodiments be employed in conjunction with the algorithm on the phone. For example, the closed loop algorithm on the pump can be used as a safety check on the closed loop algorithm on the phone. In such an embodiment, when a therapy command is received at the pump from the phone, the command can be compared to therapy as determined by the algorithm on the pump. If the commands are the same or similar (e.g., within a threshold percentage of each other) the command from the phone can be executed with the pump. If the commands are not similar (e.g., not within a threshold percentage of each other), the command from the phone may not be executed. In such a circumstance, the therapy calculated by the pump may be delivered instead and the pump may transition to using the algorithm on the pump going forward until an error message on the phone relating to accuracy of the algorithm operating on the phone is addressed. In such embodiments, the algorithm on the pump may be a more conservative algorithm that attempts to maintain glucose levels in a wider and higher range, such that when the algorithms disagree is it considered safer to deliver the dose calculated by the algorithm on the pump rather than the dose calculated by the more aggressive algorithm on the phone that may attempt to maintain the user’s glucose at lower levels.

Although the systems and methods described herein primarily focus on closed loop control algorithms operating on a phone and a pump, it should be understood that various other devices can alternatively or additionally be employed to operate closed loop algorithms. For example, a user’s smartwatch or other similar device can also store a closed loop algorithm that can be selectively activated to provide therapy commands to a pump. In some embodiments, a multi-layer algorithmic scheme can be employed in which a phone, a watch or other device and a pump each have a closed loop therapy algorithm stored thereon. The algorithms may have decreasing degrees of complexity from the pump to the watch and then the phone and be prioritized such that the phone operates the primary algorithm, the watch operates a secondary algorithm activated if the phone is unavailable and the pump operates a fallback tertiary algorithm activated if both the phone and watch are unavailable. Other algorithms can also be available with other user devices when present.

In an embodiment, a method of providing diabetes therapy can include delivering medicament to a user with an ambulatory infusion pump according to therapy commands received from a remote electronic device of a user calculated with a primary closed loop therapy algorithm based on glucose levels of a user from a continuous glucose monitoring device. It may be detected that communications between the ambulatory infusion pump and the remote electronic device have been interrupted. Glucose levels of the user can then be received from the continuous glucose monitoring device at the ambulatory infusion pump. A fallback closed loop therapy algorithm operating on the ambulatory infusion pump can be activated in response to communications between the ambulatory infusion pump and the remote electronic device being interrupted. The fallback closed loop therapy algorithm can calculate therapy commands for delivery of medicament to the user with the ambulatory infusion pump based on the glucose levels received from the continuous glucose monitoring device at the ambulatory infusion pump.

In embodiments, the fallback closed loop therapy algorithm is less complex than the primary closed loop therapy algorithm.

In embodiments, the primary closed loop algorithm utilizes one or more mathematical models that are not used by the fallback closed loop therapy algorithm.

In embodiments, the primary closed loop algorithm executes one or more mathematical calculations that are not used by the fallback closed loop therapy algorithm.

In embodiments, the primary closed loop algorithm utilizes data that is not used by the fallback closed loop therapy algorithm.

In embodiments, the primary closed loop algorithm receives information from one or more sensors that is not received by the fallback closed loop therapy algorithm.

In embodiments, the fallback closed loop therapy algorithm requires lower computational and memory capabilities to be operated than the primary closed loop therapy algorithm.

In embodiments, the fallback closed loop therapy algorithm requires less battery power to be operated than the primary closed loop therapy algorithm.

In embodiments, the fallback closed loop therapy algorithm operating on the ambulatory infusion pump can be activated in response to detecting that a battery level of the remote electronic device is below a threshold level.

In embodiments, the primary closed loop algorithm can be reactivated upon detecting that communications between the ambulatory infusion pump and the remote electronic device have been reestablished.

In an embodiment, an ambulatory infusion pump can include a pump mechanism configured to facilitate delivery of insulin to a user and a communications interface configured to receive communications from a remote electronic device. At least one processor can be configured to receive therapy commands from the remote electronic device calculated with a primary closed loop algorithm based on glucose levels of a user from a continuous glucose monitoring device. If it is detected that communications with the remote electronic device have been interrupted the glucose levels of the user from the continuous glucose monitoring device can be received and a fallback closed loop therapy algorithm can be activated in response to communications with the remote electronic device being interrupted. The fallback closed loop therapy algorithm can calculate therapy commands for delivery of medicament to the user based on the glucose levels received from the continuous glucose monitoring device.

In embodiments, the fallback closed loop therapy algorithm is less complex than the primary closed loop therapy algorithm.

In embodiments, the primary closed loop algorithm utilizes one or more mathematical models that are not used by the fallback closed loop therapy algorithm.

In embodiments, the primary closed loop algorithm executes one or more mathematical calculations that are not used by the fallback closed loop therapy algorithm.

In embodiments, the primary closed loop algorithm utilizes data that is not used by the fallback closed loop therapy algorithm.

In embodiments, the primary closed loop algorithm receives information from one or more sensors that is not received by the fallback closed loop therapy algorithm.

In embodiments, the fallback closed loop therapy algorithm requires lower computational and memory capabilities to be operated than the primary closed loop therapy algorithm.

In embodiments, the fallback closed loop therapy algorithm requires less battery power to be operated than the primary closed loop therapy algorithm.

In embodiments, the at least one processor can be further configured to activate the fallback closed loop therapy algorithm operating on the ambulatory infusion pump in response to detecting that a battery level of the remote electronic device is below a threshold level.

In embodiments, the at least one processor can be further configured to reactivate the primary closed loop algorithm upon detecting that communications between the ambulatory infusion pump and the remote electronic device have been reestablished.

In embodiments, an ambulatory infusion pump system can include a remote electronic device and an ambulatory infusion pump. The remote electronic device can be configured to receive glucose levels of a user from a continuous glucose monitoring device and to calculate therapy commands with a primary closed loop algorithm based on the glucose levels. The ambulatory infusion pump can be configured to receive the therapy commands from the remote electronic device and to deliver insulin to the user according to the therapy commands. The ambulatory infusion pump can be further configured to detect that communications with the remote electronic device have been interrupted. In response, the glucose levels of the user can be received from the continuous glucose monitoring device at the ambulatory infusion pump and a fallback closed loop therapy algorithm can be activated. The fallback closed loop therapy algorithm can calculate therapy commands for delivery of medicament to the user based on the glucose levels received from the continuous glucose monitoring device.

In embodiments, the fallback closed loop therapy algorithm is less complex than the primary closed loop therapy algorithm.

In embodiments, the fallback closed loop therapy algorithm requires lower computational and memory capabilities to be operated than the primary closed loop therapy algorithm.

In embodiments, the fallback closed loop therapy algorithm requires less battery power to be operated than the primary closed loop therapy algorithm.

In embodiments, the ambulatory infusion pump is further configured to deactivate the fallback closed loop therapy algorithm and to receive therapy commands from the remote electronic device upon detecting that communications between the ambulatory infusion pump and the remote electronic device have been reestablished.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to producenumerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Also incorporated herein by reference in their entirety are commonly owned U.S. Patent Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425; 12,214,159; and 12,357,751 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; ; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924; 2024/0399051; 2024/408303; 2024/0416032; 2024/0416033; 2025/0099674; 2025/0099675 2025/0099678; 2025/0099679; and 2025/0108162 and commonly owned U.S. Patent Applications Nos. 17/368,968; 17/896,492; 18/398,543; 18/962,169; 19/003,140; 19/003,164; 19/119,554; 19/134,333; 19/205,083; 19/220,426; 19/221,933; 19/225,150; and 19/252,256.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A method of providing diabetes therapy, comprising:
delivering medicament to a user with an ambulatory infusion pump according to therapy commands received from a remote electronic device of a user calculated with a primary closed loop therapy algorithm based on glucose level readings of a user from a continuous glucose monitoring device;
detecting that communications between the ambulatory infusion pump and the remote electronic device have been interrupted;
receiving the glucose level readings of the user from the continuous glucose monitoring device at the ambulatory infusion pump; and
activating a fallback closed loop therapy algorithm operating on the ambulatory infusion pump in response to communications between the ambulatory infusion pump and the remote electronic device being interrupted, the fallback closed loop therapy algorithm calculating therapy commands for delivery of medicament to the user with the ambulatory infusion pump based on the glucose level readings received from the continuous glucose monitoring device at the ambulatory infusion pump.

2. The method of claim 1, wherein the fallback closed loop therapy algorithm is less complex than the primary closed loop therapy algorithm.

3. The method of claim 2, wherein the primary closed loop algorithm utilizes one or more mathematical models that are not used by the fallback closed loop therapy algorithm.

4. The method of claim 2, wherein the primary closed loop algorithm executes one or more mathematical calculations that are not used by the fallback closed loop therapy algorithm.

5. The method of claim 2, wherein the primary closed loop algorithm utilizes data that is not used by the fallback closed loop therapy algorithm.

6. The method of claim 2, wherein the primary closed loop algorithm receives information from one or more sensors that is not received by the fallback closed loop therapy algorithm.

7. The method of claim 1, wherein the fallback closed loop therapy algorithm requires lower computational and memory capabilities to be operated than the primary closed loop therapy algorithm.

8. The method of claim 1, wherein the fallback closed loop therapy algorithm requires less battery power to be operated than the primary closed loop therapy algorithm.

9. The method of claim 1, further comprising activating the fallback closed loop therapy algorithm operating on the ambulatory infusion pump in response to detecting that a battery level of the remote electronic device is below a threshold level.

10. The method of claim 1, further comprising reactivating the primary closed loop algorithm upon detecting that communications between the ambulatory infusion pump and the remote electronic device have been reestablished.

11. An ambulatory infusion pump, comprising:
a pump mechanism configured to facilitate delivery of insulin to a user;
a communications interface configured to receive communications from a remote electronic device; and
at least one processor configured to:
receive therapy commands from the remote electronic device calculated with a primary closed loop algorithm based on glucose level readings of a user from a continuous glucose monitoring device;
detect that communications with the remote electronic device have been interrupted;
receive the glucose level readings of the user from the continuous glucose monitoring device; and
activate a fallback closed loop therapy algorithm in response to communications with the remote electronic device being interrupted, the fallback closed loop therapy algorithm calculating therapy commands for delivery of medicament to the user based on the glucose level readings received from the continuous glucose monitoring device.

12. The ambulatory infusion pump of claim 11, wherein the fallback closed loop therapy algorithm is less complex than the primary closed loop therapy algorithm.

13. The ambulatory infusion pump of claim 11, wherein the fallback closed loop therapy algorithm requires lower computational and memory capabilities to be operated than the primary closed loop therapy algorithm.

14. The ambulatory infusion pump of claim 11, wherein the fallback closed loop therapy algorithm requires less battery power to be operated than the primary closed loop therapy algorithm.

15. The ambulatory infusion pump of claim 11, wherein the at least one processor is further configured to reactivate the primary closed loop algorithm upon detecting that communications between the ambulatory infusion pump and the remote electronic device have been reestablished.

16. An ambulatory infusion pump system, comprising:
a remote electronic device configured to receive glucose level readings of a user from a continuous glucose monitoring device and to calculate therapy commands with a primary closed loop algorithm based on the glucose level readings; and
an ambulatory infusion pump configured to receive the therapy commands from the remote electronic device and to deliver insulin to the user according to the therapy commands, wherein the ambulatory infusion pump is further configured to:
detect that communications with the remote electronic device have been interrupted;
receive the glucose level readings of the user from the continuous glucose monitoring device; and
activate a fallback closed loop therapy algorithm in response to communications with the remote electronic device being interrupted, the fallback closed loop therapy algorithm calculating therapy commands for delivery of medicament to the user based on the glucose level readings received from the continuous glucose monitoring device.

17. The system of claim 16, wherein the fallback closed loop therapy algorithm is less complex than the primary closed loop therapy algorithm.

18. The system of claim 16, wherein the fallback closed loop therapy algorithm requires lower computational and memory capabilities to be operated than the primary closed loop therapy algorithm.

19. The system of claim 16, wherein the fallback closed loop therapy algorithm requires less battery power to be operated than the primary closed loop therapy algorithm.

20. The system of claim 16, wherein the ambulatory infusion pump is further configured to deactivate the fallback closed loop therapy algorithm and to receive therapy commands from the remote electronic device upon detecting that communications between the ambulatory infusion pump and the remote electronic device have been reestablished.
